(19) United States
(12) Patent Application Publication
(10) Pub. No.: US 2011/0201898 A1
(43) Pub. Date: Aug. 18, 2011

(54) Wireless healthcare smart grid

(76) Inventors: David S. Benco, Winfield, IL (US); Mark A. Ristich, Naperville, IL (US)

(21) Appl. No.: 12/658,877

(22) Filed: Feb. 17, 2010

(51) Int. Cl. A61B 5/00; G08B 1/08

(52) U.S. Cl. 600/300; 340/539.1

(57) ABSTRACT

One implementation encompasses an apparatus, which comprises: at least one sensor; at least one controllable element; and at least one server operatively coupled to the at least one sensor and to the at least one controllable element, the at least one server configured to receive input data from the at least one sensor, and the at least one server configured to control the at least one controllable element based on the input data received from the at least one sensor.

### TECHNICAL FIELD

The invention relates generally to telecommunication systems, and, in particular, to a wireless infrastructure that is suitable for supporting health care.

### BACKGROUND

Healthcare costs continue to rise globally, at a pace that exceeds the rate of inflation and the growth of GDP for most countries. In addition, society is seeking ways to reduce the carbon footprint of all citizens. In parallel, wireless communication is becoming more robust in terms of available bandwidth, low latency, and reliability, as evidenced by the 4G and LTE (Long Term Evolution) technologies being implemented today. Yet these recent advances in wireless communications have not substantially improved personal healthcare, have not helped to curb healthcare costs (by utilizing computers vs. humans for menial health record collection and management), and have not reduced the carbon footprint associated with the delivery of healthcare (by reducing travel requirements, since health-related services can be delivered remotely in many cases).

The specific problem at hand is the lack of a wireless infrastructure suitable for healthcare to support such capabilities as remote monitoring, early warning detection, alerting, encryption, location identification, and in general modernize the medical system by having all records (including dynamic real-time measurements) readily accessible online by both patients and their providers, etc.

Furthermore, legacy information systems of most health care players, such as insurers, hospitals, and physicians, are not ready to be used with networks. Information systems departments in most health care organizations are not Web-oriented. Also, health care presently has many standards for electronic communications and transactions.

### SUMMARY

One implementation encompasses an apparatus, which comprises: at least one sensor; at least one controllable element; and at least one server operatively coupled to the at least one sensor and to the at least one controllable element, the at least one server configured to receive input data from the at least one sensor, and the at least one server configured to control the at least one controllable element based on the input data received from the at least one sensor.

Another implementation encompasses a system, which comprises: at least a first operational level having at least a plurality of sensors and controllable elements, a second operational level having at least a plurality of communication and control devices, and a third operational level having a plurality of servers and databases (also referred to as databases, the first operational level operatively coupled to the second operational level, and the second operational level operatively coupled to the third operational level; the sensors and controllable elements being activatable and configurable to receive and send data, at least one of a first portion of the sensors and controllable elements associated with a predetermined entity, and a second portion of the sensors and controllable elements associated with an environment of the predetermined entity; the communication and control devices being programmable and configurable to send and receive data and control the sensors and controllable elements; and the servers configured to activate and communicate with the sensors and controllable elements via the communication and control devices, and the databases configured to store information relative to at least the plurality of sensors and controllable elements.

A further implementation encompasses a method, which comprises: sensing at least one parameter and forming first data indicative of the at least one parameter; wirelessly sending the first data to a server; analyzing the first data at the server; and in response to the analysis, the server wirelessly effecting, in real time, at least one of changing a mode of a first sensor, and activating a further sensor for sensing further parameters and forming second data indicative of the further parameters.

Another implementation encompasses an apparatus, which comprises: a server configured to wirelessly receive at least first data indicative of at least one parameter relative to an entity; the server configured to analyze the received data and to formulate a response based on the analyzed data; and the server configured to wirelessly effect, in real time and according to the response, at least one of changing a mode of sensing the entity, and activating further sensing relative to the entity to produce further data indicative of at least one further parameter relative to the entity.

### DESCRIPTION OF THE DRAWINGS

The features of the embodiments of the present method and apparatus are set forth with particularity in the appended claims. These embodiments may best be understood by reference to the following description taken in conjunction with the accompanying drawings, in the several figures of which like reference numerals identify like elements, and in which:

FIG. 1 depicts an embodiment according to the present method and apparatus in very basic terms.

FIG. 2 depicts a sensor and a controllable element that may be a unitary sensor/controllable element.

FIG. 3 depicts another embodiment according to the present method and apparatus.

FIG. 4 depicts an embodiment of a method according to the present method.

FIG. 5 depicts the network architecture for a wireless healthcare smart grid.

FIG. 6 depicts one example according to present the method and apparatus.

FIG. 7 depicts another example according to present the method and apparatus.

### DETAILED DESCRIPTION

Best current practice for remote monitoring and delivery of healthcare is largely limited to wired connections (e.g., modems and telephone lines, cable TV internet connections, etc.). While this is acceptable for homebound patients, it is inadequate for most people. Furthermore, the existing practice adopts a view of treatment vs. prevention. A better system would be one in which the health of mobile, active people can be enhanced, rather than merely treating illness after it has occurred. The wireless alternatives currently in use are limited to a localized WiFi “hot spot” as might be found in a given area of a hospital for example. Furthermore, the existing solutions are difficult to scale to hundreds of thousands or millions of people.

Embodiments of the present method and apparatus provide a scalable wireless infrastructure for healthcare applications. Specifically, this means utilizing the upcoming 4G network technologies (e.g., LTE) of wireless service providers for ubiquitous access, along with the network elements needed for health/fitness monitoring (via smart monitoring devices), data upload and storage, access security, healthcare applications server and database, etc.

Embodiments of the present method and apparatus provide a unique way to offer real-time health-related services to a very broad range of society in a cost-effective and environment-friendly (eco-friendly, green) manner.

Embodiments of the present method and apparatus provide a number of advantages. The embodiments enable cost-effective and eco-friendly delivery of a broad range of healthcare services using wireless technologies. Current wireless healthcare applications are localized to a small area. The embodiments also provide compelling application enablement in a huge, global vertical market space (healthcare).

Embodiments of the present method and apparatus may be utilized in both 3G and 4G networks, including CDMA, UMTS, and LTE, and is therefore applicable to all major wireless operators and healthcare vertical market incumbents. The embodiments are also compatible with both IMS and pre-IMS network configurations. They also directly address service providers' desire for applications that utilize wireless networks for “machine-to-machine” communications, thus expanding the use of 4G wireless networks.

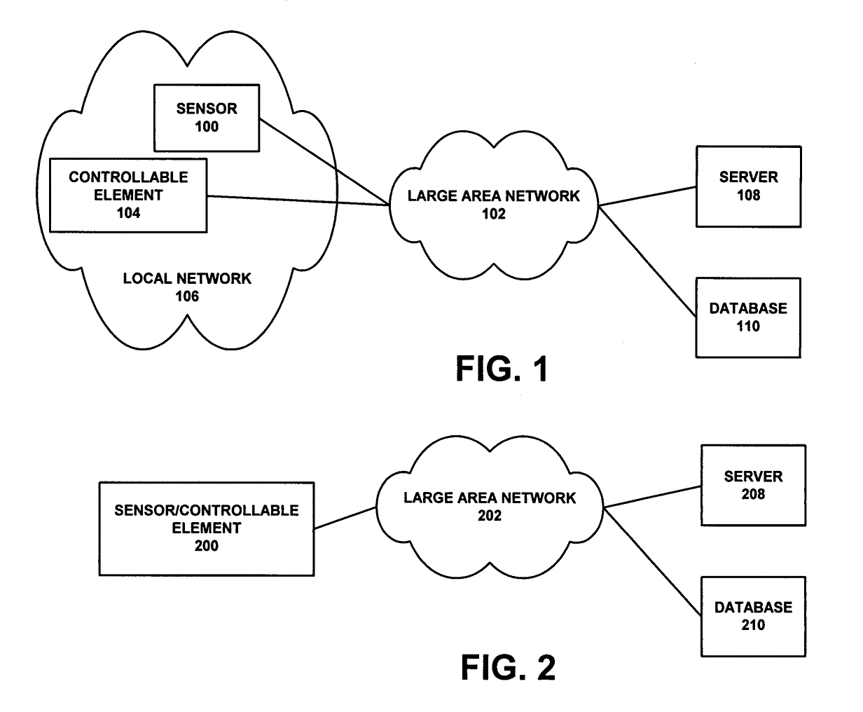

FIG. 1 depicts an embodiment according to the present method and apparatus in very basic terms. In this embodiment at least one sensor 100, such as a heart rate monitor, are operatively coupled to at least one large area network 102. Such a large area network 102 may be, for example, an IP network, such as the Internet. A controllable element 104, such as an activatable air pollution sensor, is also operatively coupled to the large area network 102. The sensor 100 and the controllable element 104 are part of a local network 106.

At least one server 108 is operatively coupled to the at least one large area network 102, the at least one server 108 configured to receive input data from the at least one sensor 100, and the at least one server 108 configured to control the at least one controllable element 104 based on the input data received from the at least one sensor 100.

The embodiment further comprises at least one database 110 that is operatively coupled to the at least one large area network 102. The at least one database 110 is configured to store information relative to the at least one sensor 100, the at least one controllable element 104, and the at least one server 108. Alternatively, the database 110 is directly coupled to the server 108, or the database 110 may be part of the server 108.

FIG. 2 depicts an embodiment in which a sensor and a controllable element are a unitary sensor/controllable element 200. The unitary sensor/controllable element 200 is operatively coupled to the large area network 202 in addition to the server 208 and the database 210.

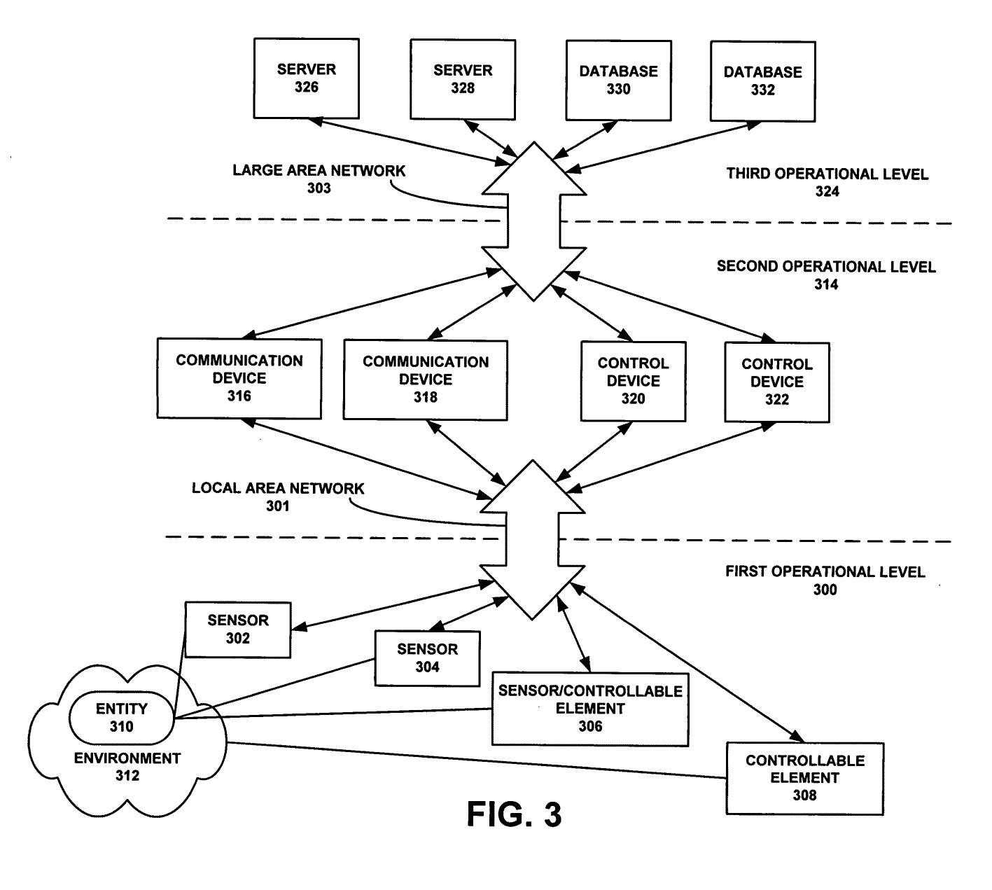

FIG. 3 depicts another embodiment according to the present method and apparatus, which is displayed in terms of operational levels. FIG. 3 is a functional diagram in which the functional elements as shown can be directly or indirectly coupled. The functional elements can also be configured from single devices or multiple devices. This embodiment has at least a first operational level 300 having one or more sensors 302, 304, unitary sensor/controllable element 306, and a controllable element 308. Each of these is operatively coupled respectively as shown to an entity 310, such as a person, and/or to an environment 312 in which the person (entity 310) is currently located. Thus, the first operational level 300 interfaces directly with the entity 310 and the environment 312.

The embodiment further has a second operational level having one or more communication devices 316, 318 and control devices 320, 322. This second operational level therefore functions as a communication level.

The embodiment also has a third operational level 324 having one or more servers 326, 328 and databases 330, 332. Processing and decision making take place in the third operation level.

The first operational level 300 is operatively coupled to the second operational level 314 by, for example, a home area network, or a body area network. That is, the first operational level 300 is operatively coupled to the second operational level 314 by a local area network 301. The second operational level 314 is operatively coupled to the third operational level 324 by, for example, a 4G network or the Internet. That is, the second operational level 314 is operatively coupled to the third operational level 324 by a large area network 303.

In the depicted embodiment the sensors 302, 304, the unitary sensor/controllable element 306 and the controllable element 308 are activatable and configurable to receive and send data. In some embodiments at least one of a first portion of the sensors and controllable elements, such as sensors 302, 304, 306 are associated with the entity 310, and the controllable element 308 are associated with an environment 312 in which the entity 310 is currently located.

In the depicted embodiment the communication and control devices 316, 318, 320, 322 are programmable and configurable to send and receive data, as well as, to control the sensors 302; 304, the unitary sensor/controllable element 306, and the controllable element 308. The servers 326, 328 are configured to activate and communicate with the sensors 302, 304, the unitary sensor/controllable element 306, and the controllable element 308 via the communication and control devices 316, 318, 320, 322. The databases 330, 332 are configured to store information relative to the sensors 302, 304, the unitary sensor/controllable element 306, and the controllable element 308, and the servers 326, 328.

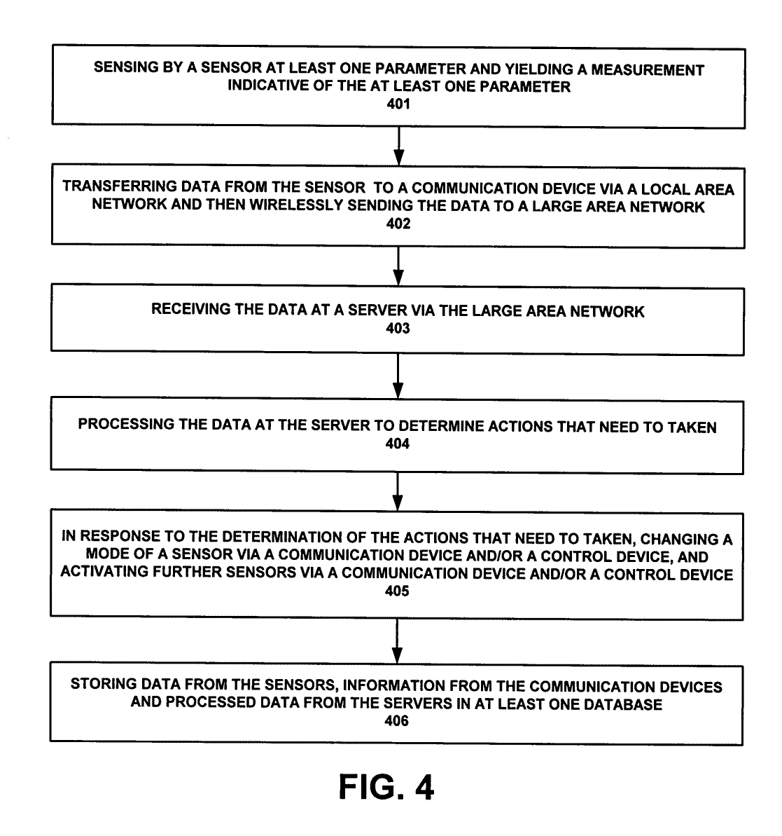

FIG. 4 depicts an embodiment of a method according to the present specification. The method may have the following steps. In a first step 401 a sensor (e.g. 302, 304, 306) senses at least one parameter and yields a measurement, such as data in a predetermined format, indicative of the at least one parameter. The sensor 302, 304, 306 can be any one of many different types for measuring, for example, heart rate, body temperature, caloric intake, medication levels, etc. of a person, or for measuring different environmental conditions, such as temperature, humidity, etc., which currently affect the person. The data defines a measurement of the sensor, for example, it can be a numerical value of a temperature of the person at a certain point in time.

In a next step 402 the data is transferred from the sensor to a large area network. More specifically, in one example, the data is transferred from the sensor to a communication device 316, 318 via a local area network 301 and then wirelessly sent to a large area network 303. In a next step 403 the data is received at a server 326, 328 via the large area network 303. In a next step 404 the server 326, 328 processes the data to determine actions that need to taken, such as measuring other parameters relative to the entity 310. In a next step 405, in response to the determination of the actions that need to taken, sensors 302, 304, 306 are activated and deactivated. That is, actions are taken, such as, changing a mode of a sensor 302, 304, 306 via a communication device 316, 318 and/or a control device 320, 322, and activating further sensors 302, 304, 306 via a communication device 316, 318 and/or a control device 320, 322. The method may also have the step 406 of storing data from the sensors 302, 304, 306, information from the communication devices 316, 318, and processed data from the servers 326, 328 in at least one database 330, 332.

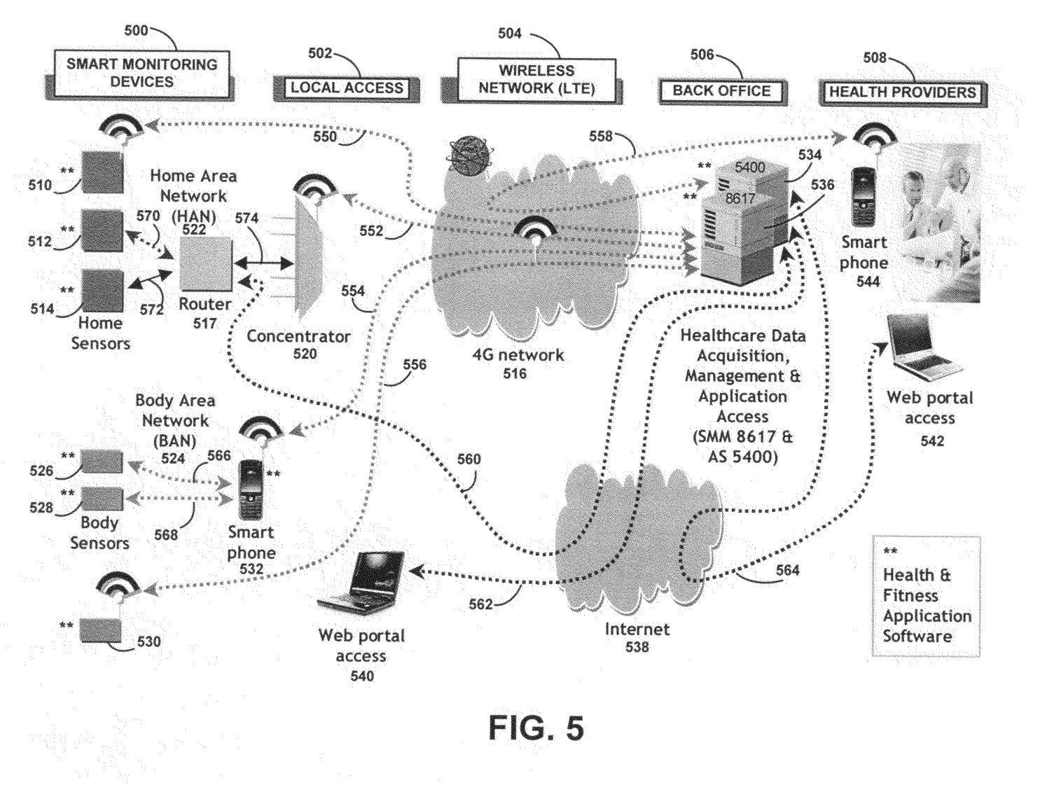

FIG. 5 depicts one example of network architecture for a wireless healthcare smart grid. An end-to-end solution is illustrated, from smart monitoring devices to wireless transmission of data, collection and storage of data, and access by both individuals and healthcare providers.

In the depicted embodiment the network architecture is broken down into smart monitoring devices 500, local access 502, wireless network (LTE) 504, back office 506, and healthcare providers 508. The monitoring devices 500 are elements of a first operational level, devices for local access 502 are elements of a second operational level, and the back office 506 and health providers 508 are elements of a third operational level. These elements are respectively coupled by local area networks, such as home area network 522 and body area network 524, and by large area networks 504, such as 4G network 516 and the Internet 538.

In the depicted embodiment home sensors 510, 512, 514 are operatively coupled to a 4G network 516 via a router 517 and a concentrator 520, and are part of a home area network 522. A body area network 524 has body sensors 526, 528, and a further sensor 530. Sensors 526, 528 are operatively coupled to the 4G network 516 via smart phone 532.

In the depicted embodiment server 534 and database 536 are operatively coupled to the 4G network 516, and to the Internet 538. Web portal access 540, 542 have access to the server 534 and the database 536 via the Internet 538. Service providers also access the server 534 and the database 536 via a smart phone 544 and the 4G network 516.

In the depicted embodiment communication paths 550, 552, 554, 556, 558 represent wireless communication over LTE or other 4G networks 516. Communication paths 560, 562, 564 represent communication over the (wired) Internet 538. Communication paths 566, 568 represent communication over a wireless Body Area Network (BAN) 524 (or Personal Area Network (PAN)) using a communications protocol such as Bluetooth (IEEE 802.15.1). Communication path 570 represents communication over a wireless Home Area Network (HAN) 522 using a communications protocol such as WiFi (IEEE 802.11g, IEEE 802.11b, IEEE 802.11n, etc.). Communication paths 572, 574 represent communication over a wired (copper or optical) Local Area Network (LAN) 522 using a communications protocol such as Ethernet (IEEE 802.3, IEEE 802.3u, etc.).

In the depicted embodiment the concentrator 520 is a functional unit that permits a common path to handle more data sources than there are channels currently available within the path. The concentrator 520 may be used primarily to cover facilities where mobility is restricted, such as clinics, assisted living facilities, even certain sections of hospitals. The advantage of the concentrator 520 is that it interfaces to the LTE network 516, thus enabling rapid communication with the primary care physician or health providers 508, specialists, next of kin, etc. in the event a medical condition change warrants such communication. The low latency of the LTE network 516 also provides a more robust connection for the healthcare sensors 510, 512, 514 by eliminating dependence on the less reliable Internet 538. This is particularly desirable given the generally more serious medical requirements of this group. Relevant communication paths are 574 and 552, for example.

In the depicted embodiment home sensors 510, 512, 514 comprise environmental quality indicators which can measure and report air pollutants, pollen and mold counts, humidity, etc. Full-featured home sensors 510, 512, 514 are capable of controlling other devices, such as air filters, air conditioners, etc. in response to an external trigger. They are also remotely controllable to enter different modes of operation. Home sensors 510, 512 514 can utilize a Home Area Network (HAN) 522 such as WiFi (e.g., IEEE 802.11g) or be hardwired to a router 517 for Internet connectivity. Permanently installed home sensors 510, 512 514 normally use a wired connection for connectivity to the network 522, although specialized monitoring equipment for temporary use relies more often on wireless connectivity. Relevant communication paths are 550, 570 and 572, for example.

In the depicted embodiment body sensors 526, 528, 530 are capable of measuring heart rate, body temperature, caloric intake, medication levels, etc. Casual applications (e.g., fitness, diet, etc.) often utilize a Body Area Network (BAN) 524 to communicate with a smart phone 532 via NFC (Near Field Communication) protocols such as Bluetooth. Wireless communication to/from the network 516 is handled by the smart phone 532. More demanding applications (e.g., location/motion detectors for senior citizens living alone, diagnostic test sensors for sleep disorders, medication reminders. etc.) utilize wireless connectivity. Relevant communication paths are 556, 566 and 568, for example.

In the depicted embodiment, in addition to the wireless connectivity of the smart phone 532 for body sensor reporting, the smart phone 532 is an applications access device in itself (e.g., workout summary, diet progress, prescription refill reminders, etc.). Relevant communication paths are 566, 568 and 554, for example.

In the depicted embodiment the wireless network 516 utilizes a 4G technology such as LTE (Long Term Evolution), due to its high bandwidth, low latency, and improved efficiency (i.e., lower cost). LTE allows the healthcare wireless smart grid to scale from the dozens of monitoring devices supported by a WiFi hotspot to the millions of people in a city or a region. It enables centralization of home and body sensor data collection, management and access. It removes the restrictions of home or other fixed locations for healthcare applications. It enables rich communication, such as video calling, with minimal delays in response to alerts triggered by the analysis of home and/or body sensors. Because the network 516 is global in reach, it offers instant connectivity to specialists, emergency services, next of kin, etc. for those individuals whose medical condition warrants close attention. Relevant communication paths are 550, 552, 554, 556 and 558, for example.

In the depicted embodiment for sensors 510, 512, 514, 526, 528, 530, an SMM 8617 device is one example of a logical network interface to home and body sensors. Smart Metering Management (SMM) is a utility-provider software application for real-time monitoring of meters in an IP environment. It understands the protocols and capabilities of each type of sensor. Sensor application software in the SMM 8617 is capable of triggering alternate sensor behavior ranging from changing the type of measurements taken to triggering sensor-connected external devices. The SMM 8617 is the repository for all sensor data. Recent data is available on local disk and earlier data is available via network access to archived storage. The SMM 8617 offers APIs so that new sensors can be introduced quickly into the market by third parties.

In the depicted embodiment for server 534, an AS 5400 device is one example of a healthcare applications server. The AS 5400 offers web portal access to health care providers 508, individuals, physicians, specialists, hospitals, clinics, etc. via applications customized to the needs of each group of stakeholders. The AS 5400 obtains data from the SMM 8617 as required. Security and access restrictions are enforced by the AS 5400. Ancillary (non-sensor) data (such as individual address, contact information, etc.) is stored on the AS 5400. The AS 5400 offers a rich set of APIs to allow rapid development of new health and fitness applications using Web-based abstractions such as Web Services Description Language (WSDL).

Access to an individual's own medical information is a key advantage of the wireless healthcare smart grid. A patient can have access to his medical information using his own smart phone, for example. Also, such access can be obtained, for example, by use of web portal access 540, 542 and respective communication paths 562, 564. Access ranges from fitness and diet progress (trends, charts, goals, history, etc.) to written instructions from recent doctor visits. Many times a person must rely on scribbled notes or on memory or may have misplaced the physician instructions for follow-up care after a doctor visit. Embodiments of the present method and apparatus eliminate these problems. Links to explanation of medical terms, explanation of parameters and levels of test results allow the patient to be much more engaged in his/her health and fitness.

Combining fitness equipment with smart phone capabilities allows individuals an easy way to record their daily weight, resting heart rate, etc. This baseline information is invaluable to physicians as an overall indication of general health.

Secure Internet access to patient sensor data and medical history is provided, by for example using portal access 540, 542 or smart phone 544, to healthcare professionals, hospitals, insurance companies, patients etc. via, for example, the AS 5400 applications that are specific to each stakeholder type. In this way, privacy and security of patient data is preserved. Access to records can by “pull” type of access, or by “push” type of access. The LTE network 516 is capable of providing alerts to Internet-connected devices as well as smart phones, wired phones, etc. Depending on the specific reasons for the alert, the appropriate supplemental data can be provided in the context of the alert, which better prepares medical staff, gives early notification and background information to specialists, etc. The transfer of rich data (video, images, summary charts, etc.) over the LTE network 516 to capable devices reduces patient waiting times and greatly improves the efficiency of the physician. The LTE network 516 also provides ready real-time communication between physician and patient before patient arrival.

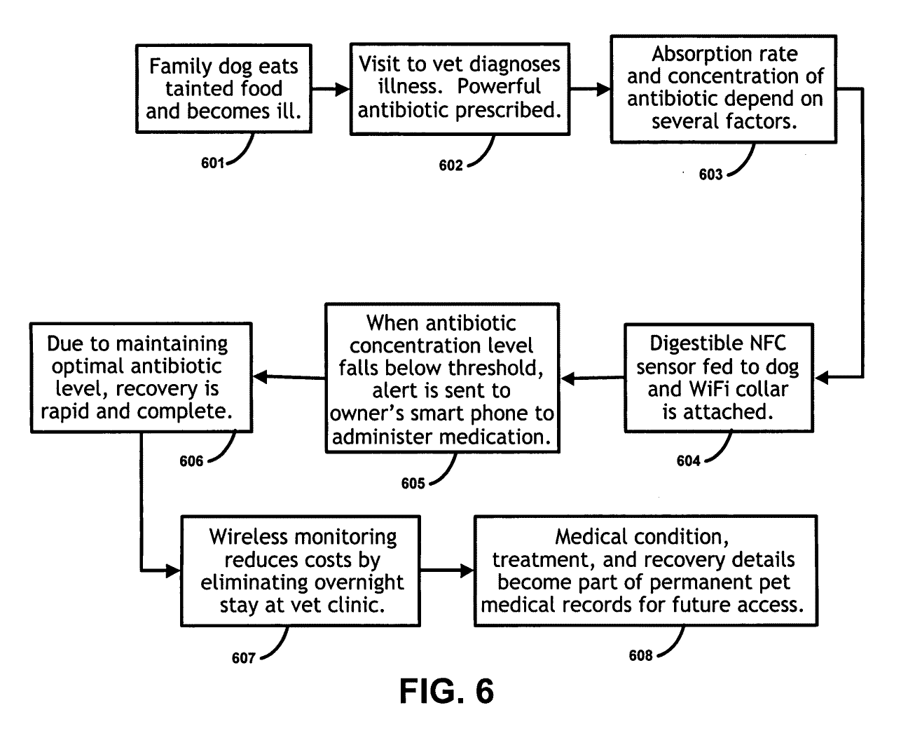

FIG. 6 depicts one example according to present the method and apparatus. In this example a family dog eats tainted food and becomes ill (step 601). At an animal clinic a veterinarian diagnoses the illness, and a powerful antibiotic is prescribed (step 602). An important consideration in the use of such an antibiotic is that the absorption rate and concentration of the antibiotic depend on several factors, such as hydration, activity level, weight, and appetite (step 603). A body sensor 526, 528, 530, such as a digestible NFC (Near Field Communication) sensor, is fed to the dog and a WiFi collar is attached to the dog (step 604). Near field communication between the WiFi collar and the NFC sensor is a very short-range (max. 20 cm, but typically only a few cm), wireless point-to-point interconnection technology, which is based on inductive coupling.

When the antibiotic concentration level falls below a predetermined threshold, an alert is sent to the owner's smart phone for medication administration to the dog (step 605). By maintaining an optimal antibiotic level, recovery is rapid and complete (step 606). The wireless monitoring reduces costs by eliminating overnight stay at a veterinarian clinic (step 607). Medical condition, treatment, and recovery details become part of permanent pet medical records for future access (step 608).

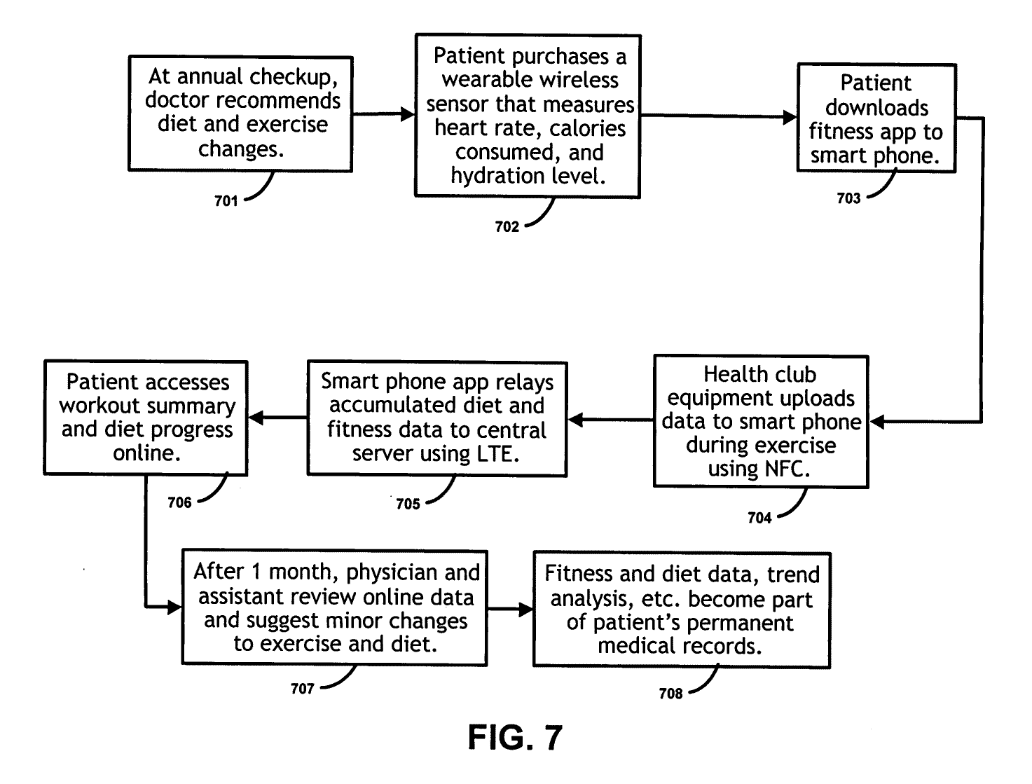

FIG. 7 depicts another example according to present the method and apparatus.

At an annual checkup, a doctor recommends diet and exercise changes for a patient (step 701). The patient purchases a wearable wireless sensor 526, 528, 530 that measures, for example, heart rate, calories consumed, and hydration level (step 702). The patent then downloads one or more fitness apps (applications) to a smart phone 532 (step 703).

While exercising at a health club, health club equipment sensors 526, 528 upload data, such as body weight, calories expended, duration, and respiratory rate, to the smart phone 532 using NFC (Near Field Communication) (step 704). Accumulated diet and fitness data is relayed by the smart phone 532 to a central server 534 via the LTE network 516 (step 705).

The patient can then access workout summary and diet progress online via, for example, web portal access 540 or smart phone 532 (step 706). At some point in time, for example after one month, the physician may review the online data and suggest minor changes to the exercise and diet program, which are automatically updated on the smart phone 532 (step 707). The physician may also modify the exercise and diet program, or change the parameters that are measured. The fitness and diet data, trend analysis, etc. become part of a patient's permanent medical record (step 708).

The present apparatus in one example may comprise a plurality of components such as one or more of electronic components, hardware components, and computer software components. A number of such components may be combined or divided in the apparatus.

The present apparatus in one example may employ one or more computer-readable signal-bearing media. The computer-readable signal-bearing media may store software, firmware and/or assembly language for performing one or more portions of one or more embodiments. The computer-readable signal-bearing medium for the apparatus in one example may comprise one or more of a magnetic, electrical, optical, biological, and atomic database medium. For example, the computer-readable signal-bearing medium may comprise floppy disks, magnetic tapes, CD-ROMs, DVD-ROMs, hard disk drives, and electronic memory. In another example, the computer-readable signal-bearing medium may comprise a modulated carrier signal transmitted over a network comprising or coupled with the apparatus, for instance, one or more of a telephone network, a local area network (“LAN”), a wide area network (“WAN”), the Internet, and a wireless network.

The steps or operations described herein are just exemplary. There may be many variations to these steps or operations without departing from the spirit of the invention. For instance, the steps may be performed in a differing order, or steps may be added, deleted, or modified.

Although exemplary implementations of the invention have been depicted and described in detail herein, it will be apparent to those skilled in the relevant art that various modifications, additions, substitutions, and the like can be made without departing from the spirit of the invention and these are therefore considered to be within the scope of the invention as defined in the following claims.

We claim:

1. An apparatus, comprising:
at least one sensor;
at least one controllable element; and
at least one server operatively coupled to the at least one sensor and to the at least one controllable element, the at least one server configured to receive input data from the at least one sensor, and the at least one server configured to control the at least one controllable element based on the input data received from the at least one sensor.

2. The apparatus according to claim 1, wherein the at least one sensor is wirelessly operatively coupled to the at least one server.

3. The apparatus according to claim 1, wherein the at least one controllable element is wirelessly operatively coupled to the at least one server.

4. The apparatus according to claim 1, wherein the at least one sensor is part of the at least one controllable element.

5. The apparatus according to claim 1, wherein the at least one server is operatively coupled to the at least one sensor and the at least one controllable element via at least one large area network, and wherein the at least one sensor and the at least one controllable element are operatively coupled to the at least one large area network via a local area network.

6. The apparatus according to claim 5, wherein the local area network is one of a home area network, and a body area network.

7. The apparatus according to claim 1, wherein the apparatus further comprises at least one database operatively coupled to the at least one server, the at least one database configured to store information relative to the at least one sensor, the at least one controllable element, and the at least one server.

8. The apparatus according to claim 1, wherein the at least one sensor and/or the at least one controllable element is operatively coupled to the at least one server via a communication device.

9. The apparatus according to claim 8, wherein the communication device is one of a programmable device, a smart phone, and a computer.

10. The apparatus according to claim 8, wherein the apparatus is configure for real time interaction between the at least one sensor, the at least one controllable element, and the at least one server.

11. A system, comprising:
at least a first operational level having one or more sensors and controllable elements, a second operational level having one or more communication and control devices, and a third operational level having one or more servers and databases, the first operational level operatively coupled to the second operational level, and the second operational level operatively coupled to the third operational level;
the sensors and controllable elements being activatable and configurable to receive and send data, at least one of a first portion of the sensors and controllable elements associated with a predetermined entity, and a second portion of the sensors and controllable elements associated with an environment in which the predetermined entity is currently located;
the communication and control devices being programmable and configurable to send and receive data and control the sensors and controllable elements; and
the servers configured to activate and communicate with the sensors and controllable elements via the communication and control devices, and the databases configured to store information relative to at least the plurality of sensors and controllable elements.

12. The system according to claim 11, wherein the first operational level is operatively coupled to the second operational level via a local area network, and wherein the second operational level is operatively coupled to the third operational level via a large area network.

13. The system according to claim 11, wherein the servers are configured to download one of a plurality of different applications to the communication and control devices, and to the sensors and controllable elements as a function of received data from the sensors and controllable elements.

14. The system according to claim 11, wherein the system is structured such that activating and configuring of the sensors and controllable elements to receive and send data, and programming and configuring the communication and control devices to send and receive data for the sensors and controllable elements occurs in real time.

15. The system according to claim 11, wherein at least one of data from the sensors and controllable elements, information from the communication and control devices, and information from the servers are stored in the databases.

16. The system according to claim 11, wherein the system is configured for dynamic changing of data requested from the sensors and controllable elements based on data received from the sensors and controllable elements.

17. The system according to claim 11, wherein the sensors and controllable elements measure at least one of biological data relative to a biological entity, and environmental data relative to the biological entity, and wherein the biological data and the environmental data are stored in at least one medical record for the biological entity in a respective database.

18. A method, comprising:
receiving first data indicative of the at least one parameter;
analyzing the first data; and
in response to the analysis, wirelessly effecting, in real time, at least one of changing a mode of a first sensor, and activating a further sensor for sensing further parameters and forming second data indicative of the further parameters.

19. The method according to claim 18, wherein the method further comprises storing at least one of first and second data in at least one database.

20. The method according to claim 18, wherein the method further comprises
measuring at least one of biological data relative to a biological entity, and environmental data relative to the biological entity; and
storing the biological data and the environmental data in at least one medical record for the biological entity in a respective database.

21. An apparatus, comprising:
a server configured to wirelessly receive at least first data indicative of at least one parameter relative to an entity;
the server configured to analyze the received data and to formulate a response based on the analyzed data; and
the server configured to wirelessly effect, in real time and according to the response, at least one of changing a mode of sensing the entity, and activating further sensing relative to the entity to produce further data indicative of at least one further parameter relative to the entity.

22. The application according to claim 21, wherein the method further comprises storing at least one of first and further data in at least one database.

23. The application according to claim 21, wherein the entity is a biological entity, wherein the server is configured to wirelessly receive at least one of biological data relative to the entity, and environmental data relative to the entity, and wherein the biological data and the environmental data are stored in at least one medical record for the biological entity.
